(19) United States
(12) Patent Application Publication
(10) Pub. No.: US 2023/0099043 A1
(43) Pub. Date: Mar. 30, 2023

(54) MULTI-DEPLOY ZONE CONSTRAINING DEVICES AND METHODS

(71) Applicant: W. L. Gore & Associates, Inc., Newark, DE (US)

(72) Inventor: Jerry J. Stastka, Flagstaff, AZ (US)

(21) Appl. No.: 17/801,892

(22) PCT Filed: Feb. 24, 2021
(86) PCT No.: PCT/US2021/019386

§ 371 Date: Aug. 24, 2022

Related U.S. Application Data
(60) Provisional application No. 62/980,660, filed on Feb. 24, 2020.

(51) Int. Cl. A61F 2/966

(52) U.S. Cl. CPC A61F 2/966; A61F 2002/9511

(57) ABSTRACT

A removable constraining device, systems, and methods for constraining and delivering an expandable member is disclosed. The removable constraining device includes a plurality of strands interlocking to form a cover body having a length, the plurality of strands in the form of a warp knit, and the plurality of strands including at least a first set of strands and a second set of strands. The removable constraining device also includes a first release zone defined by the first set of strands of the cover body along the length of the cover body and a second release zone defined by the seconds set of strands of the cover body, the second release zone being coextensive with the first release zone along at least a portion of the length of the cover body. The removable constraining device may be released at release zones by substantially simultaneously tensioning deployment lines.

### CROSS-REFERENCE TO RELATED APPLICATION

This application is a national phase application of PCT Application No. PCT/US2021/019386, internationally filed on Feb. 24, 2021, which claims the benefit of Provisional Application No. 62/980,660, filed Feb. 24, 2020, which are incorporated herein by reference in their entireties for all purposes.

### FIELD

The present disclosure relates generally to apparatuses, systems, and methods for delivery of implantable medical devices. More specifically, the disclosure relates to apparatuses, systems, and methods that include coverings for implantable medical devices during device delivery.

### BACKGROUND

Minimally invasive delivery techniques for implantable medical devices have a variety of advantages, such as reduced trauma, risk of infection, and recovery time. Examples of implantable medical devices include stents and stent-grafts utilized to radially support, treat and/or otherwise augment tubular passages in the body, including arteries, veins, airways, gastrointestinal tracts, and biliary tracts. Additional examples of implantable medical devices include prosthetic valves (e.g., prosthetic heart valves). Transcatheter delivery is a technique for delivering such implantable medical devices, where the medical device to be delivered begins in a diametrically compressed state for delivery and then is expanded (e.g., self-expanding or manually expandable) at a treatment site in the body of a patient.

Stents, stent-grafts, prosthetic valves, filters, and other implantables may be deployed by being plastically deformed (e.g., using an inflatable balloon) or permitted to self-expand and elastically recover from a collapsed or constrained, delivery diameter to an expanded, deployed diameter.

For example, U.S. Pat. No. 6,224,627, entitled “Remotely removable covering and support,” filed Jun. 15, 1998, describes, among other things, a thin tubular multiple filament (film or fiber) structure that can hold high internal pressures. When desired, an extension of the filaments can be pulled in any direction to unfurl the structure. The structure can be useful for self-expanding stent or stent graft delivery systems, balloon dilatation catheters, removable guide wire lumens for catheters, drug infusion or suction catheters, guide wire bundling casings, removable filters, removable wire insulation, removable packaging and other applications.

### SUMMARY

According to one example (“Example 1”), a removable constraining device comprises a plurality of strands interlocking to form a cover body having a length, the plurality of strands in the form of a warp knit, and the plurality of strands including a first set of strands and a second set of strands; a first release zone defined by the first set of strands of the cover body along the length of the cover body; a second release zone defined by the seconds set of strands of the cover body, the second release zone being coextensive with the first release zone along at least a portion of the length of the cover body; a first deployment line defined by the first set of strands; and a second deployment line defined by the second set of strands, wherein the first deployment line is configured to release the cover body along the first release zone by tensioning the first deployment line and the second deployment line is configured to release the cover body along the second release zone by tensioning the second deployment line.

According to another example (“Example 2”), further to the device of Example 1, the first deployment line and the second deployment line are coupled to form a unitary deployment segment.

According to another example (“Example 3”), further to the device of any one of the preceding examples, the plurality of strands forming the cover body includes four strands.

According to another example (“Example 4”), further to the device of Example 3, two strands of the four strands comprise the first set of strands forming the first deployment line and defining the first release zone, and two remaining strands of the four strands comprise the second set of strands forming the second deployment line and defining the second release zone.

According to another example (“Example 5”), further to the device of any one of the preceding examples, the first release zone comprises a first knit row and the second release zone comprises a second knit row.

According to another example (“Example 6”), further to the device of Example 5, the first knit row includes a first plurality of knits and the second knit row includes a second plurality of knits.

According to another example (“Example 7”), further to the device of Example 6, the first plurality of knits includes a first knit and a second knit and wherein the second plurality of knits includes a corresponding first knit and a corresponding second knit.

According to another example (“Example 8”), further to the device of Example 7, the first knit interferes with deployment of the corresponding second knit when the first knit is undeployed.

According to another example (“Example 9”), further to the device of Example 7 or 8, the corresponding first knit interferes with deployment of the second knit when the corresponding first knit is undeployed.

According to another example (“Example 10”), further to the device of any one of Examples 7-9, the first knit and the corresponding first knit are positioned at substantially similar longitudes along the cover body.

According to another example (“Example 11”), further to the device of any one of the preceding examples, the first release zone and the second release zone are configured to be deployed substantially simultaneously by substantially simultaneously tensioning the first and second deployment lines.

According to another example (“Example 12”), further to the device of any one of the preceding examples, the plurality of strands comprises expanded polytetrafluorethylene.

According to another example (“Example 13”), further to the device of any one of the preceding examples, the cover is configured to provide resistance to outward expansion of a constrained medical device.

According to another example (“Example 14”), further to the device of any one of the preceding examples, each strand of the plurality of strands has matching strand properties.

According to another example (“Example 15”), further to the device of Example 14, the matching strand properties include strand thickness, strand denier, strand coefficient of friction, strand material, and strand stiffness.

According to another example (“Example 16”), further to the device of any one of the preceding examples, the first deployment line is integrally formed with one or more of the plurality of strands.

According to another example (“Example 17”), further to the device of any one of the preceding examples, the second deployment line is integrally formed with one or more of the plurality of strands.

According to another example (“Example 18”), a medical device includes an expandable member configured to radially expand from a first diameter toward a second diameter; and a knit constraining member positioned around the expandable member to constrain the expandable member at the first diameter, the knit constraining member having a first release zone configured to disengage the knit constraining member from the deployable member, a first deployment line operable to activate the first release zone, a second release zone configured to disengage the knit constraining member from the deployable member, and a second deployment line operable to activate the second release zone, wherein the constraining member is configured to disengage from the deployable member by substantially simultaneously tensioning the first deployment line and the second deployment line.

According to another example (“Example 19”), further to the device of Example 18, the constraining member comprises a first body strand and a second body strand interwoven to form the first deployment line and a third body strand and a fourth body strand interwoven to form the second deployment line.

According to another example (“Example 20”), further to the device of Example 19, the first body strand, the first deployment line, the second body strand, and the second deployment line are all interwoven.

According to another example (“Example 21”), further to the device of Example 20, the first body strand, the first deployment line, the second body strand, and the second deployment line are warp knit.

According to another example (“Example 22”), further to the device of any one of the Examples 19-21, the first body strand and the first deployment line form a first plurality of knits along at least a first portion of a longitudinal length of the constraining member, and wherein the second body strand and the second deployment line form a second plurality of knits along at least a second portion of the longitudinal length of the constraining member.

According to another example (“Example 23”), further to the device of Example 22, the first release zone comprises the first plurality of knits and the second release zone comprises the second plurality of knits.

According to another example (“Example 24”), further to the device of Example 23, the first plurality of knits are sequentially unraveled when a first threshold tension is applied across the first deployment line and wherein the second plurality of knits are sequentially unraveled when a second threshold tension is applied across the second deployment line.

According to another example (“Example 25”), further to the device of Example 24, the first deployment line is interwoven with the second deployment line and the second body strand such that the second plurality of knits are operable to unravel when corresponding knits of the first plurality of knits are unraveled by advancing the first deployment line away from the first release zone.

According to another example (“Example 26”), further to the device of Examples 24 or 25, the second deployment line is interwoven with the first deployment line and the first body strand such that the first plurality of knits are operable to unravel when corresponding knits of the second plurality of knits are unraveled by advancing the second deployment line away from the second release zone.

According to another example (“Example 27”), further to the device of any one Examples 18-26, the first deployment line and the second deployment line each include free ends, wherein the free ends of the first deployment line and the second deployment line are coupled to form a unitary deployment segment.

According to another example (“Example 28”), further to the device of any one Examples 18-27, the first release zone and the second release zone are configured to deploy substantially simultaneously.

According to another example (“Example 29”), further to the device of any one Examples 18-28, the first deployment line, the first body strand, the second deployment line, and the second body strand comprise expanded polytetrafluorethylene.

According to another example (“Example 30”), further to the device of any one Examples 18-29, the first release zone and the second release zone are configured to provide resistance to an outward expansion of the deployable member when undeployed.

According to another example (“Example 31”), further to the device of any one Examples 18-30, the implantable medical device has a radial force at a delivery diameter of the deployable member, and wherein the first deployment line and the second deployment line are configured to be removed with a deployment force applied to the first deployment line and the second deployment line; and wherein a ratio of the radial force to the deployment force is between about 100 and about 500.

According to another example (“Example 32”), further to the device of Example 31, the ratio of the radial force to the deployment force is between about 170 and about 475.

According to another example (“Example 33”), further to the device of Example 31, the ratio of the radial force to the deployment force is between about 200 and about 450.

According to another example (“Example 34”), further to the device of Example 31, the ratio of the radial force to the deployment force is between about 225 and about 425.

According to another example (“Example 35”), further to the device of any one of Examples 18-34, a ratio of deployable member delivery diameter to deployment diameter is less than 0.3.

According to another example (“Example 36”), an expandable medical device including a deployable member and a removable constraint including a plurality of interlocking strands in the form of a warp knit, wherein the removable constraint is positioned axially exterior to the deployable member and restrains the deployable member in a radially compressed orientation, wherein the plurality of interlocking strands includes a first deployment line and a second deployment line, and wherein the removable constraint is configured to be remotely removed when force is applied to the first deployment line and the second deployment line.

According to another example (“Example 37”), further to the device of Example 36, the removable constraint includes a first release zone and a second release zone.

According to another example (“Example 38”), further to the device of Example 37, the first release zone is formed at least partially of the first deployment line, and wherein the second release zone is formed at least partially of the second deployment line.

According to another example (“Example 39”), further to the device of Example 38, the first release zone is configured to sequentially unravel when tension is applied to the first deployment line, and wherein the second release zone is configured to sequentially unravel when tension is applied to the second deployment line.

According to another example (“Example 40”), further to the device of any one of Examples 36-39, the removable constraint is configured to be removed from the deployable member by substantially simultaneously applying force to the first deployment line and the second deployment line.

According to another example (“Example 41”), a method of manufacturing an expandable medical device, including compressing an expandable member radially inward; interweaving a plurality of strands including a first deployment line and a second deployment line to form a removable constraint; installing the removable constraint onto the expandable member retaining a portion of the first deployment line remote from the expandable member such that the portion of the first deployment line extends away from the removable constraint; and retaining a portion of the second deployment line remote from the expandable member such that the portion of the second deployment line extends away from the removable constraint, wherein the first deployment line and the second deployment line are operable to be tensioned resulting in partial deconstruction of the removable constraint when tensioned substantially simultaneously.

According to another example (“Example 42”), further to the method of Example 41, coupling a first proximate end of the first deployment line with a second proximate end of the second deployment line such that they form a unitary deployment segment.

According to another example (“Example 43”), further to the method of any one of Example 41 or 42, the step of interweaving a plurality of strands occurs simultaneously with the step of installing the removable constraint.

According to another example (“Example 44”), further to the method of any one of Examples 41-43, the step of interweaving the plurality of strands occurs simultaneously with compressing the expandable member such that the plurality of strands provide a compressive force to the expandable member as the plurality of strands are interwoven about the expandable member.

According to another example (“Example 45”), further to the method of any one of Examples 41-44, weakening a strand of each of the first and second deployment lines.

According to another example (“Example 46”), further to the method of any one of Examples 41-44, breaking at least one strand from each of the first and second deployment lines.

According to another example (“Example 47”), further to the method of Example 46, initializing release of a first release zone and a second release zone of the removable constraint.

According to another example (“Example 48”), further to the method of Example 47, the step of initializing release of the removable constraint includes tensioning the first and second deployment lines.

According to another example (“Example 49”), further to the method of Example 48, ceasing tensioning of the first and second deployment lines prior to the first and second release zones deconstructing around the expandable member.

According to another example (“Example 50”), a method of deploying a medical device, including positioning an expandable medical device in a patient, wherein the expandable medical device is constrained by a constraining member in a compressed configuration, wherein the constraining member includes a first release zone configured to disengage the constraining member from the expandable medical device, a first deployment line operable to activate the first release zone, a second release zone configured to disengage the constraining member from the expandable medical device, and a second deployment line operable to activate the second release zone; retaining proximal portions of the first and second deployment lines remote from the expandable medical device; and applying sufficient force to the proximal portions of the first and second deployment lines to activate the first release zone and the second release zone.

According to another example (“Example 51”), further to the method of Example 50, the step of applying sufficient force to the proximal portions of the first and second deployment lines includes simultaneously applying sufficient force to the proximal portions of the first and second deployment lines.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a delivery system having a removable constraint and an expandable member in accordance with an embodiment;

FIG. 2 is a removable constraint disposed about an expandable device in accordance with one embodiment;

FIG. 3 is a removable constraint with knit rows and various knits in accordance with one embodiment; and

FIG. 4 is a weave pattern implemented fora removable constraint in accordance with one embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

Outward radial expansion force or outward radial force generally refers to the force caused by internal forces of a device when the device is formed of a plastically deformable material and is constrained or compressed to a smaller diameter. When the device is constrained at a smaller diameter, the outward radial expansion force is such that the device exerts force on a constraint. Thus, the outward radial force may be a result of a self-expanding member. Such self-expanding members may include shape memory alloys which exert an outward radial expansion force when compressed and/or constrained. However, outward radial expansion force may also refer to other forces causing a device or member to expand radially outward such as inflation of an angioplasty balloon.

Constraining force generally refers to the force exerted by a constraining member against a device when resisting the outward radial expansion force of the device, in some embodiments a self-expanding device. The constraining force may be considered a normal force resulting from the outward radial expansion force applied against the constraining member. Stated otherwise the constraining member may exert an inward radial force on the device when constraining the device. The constraining force may be limited in some embodiments to a threshold amount until the constraining member is no longer able to resist the outward radial expansion force of a device, at which point the medical device may deploy due to the outward radial expansion force overcoming the constraining force of the constraining member and thus causes the constraining member to disengage.

Deployment force generally refers to the force required to deploy the medical device by disengaging the constraining member. In some embodiments the deployment force is the force required to tension an activation line of a constraining member in order to activate disengagement of the constraining member from the medical device.

For reference, the term “circumference” is not meant to require a circular cross-section, and is instead to be understood broadly to reference an outer surface or dimension of the removable constraint.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

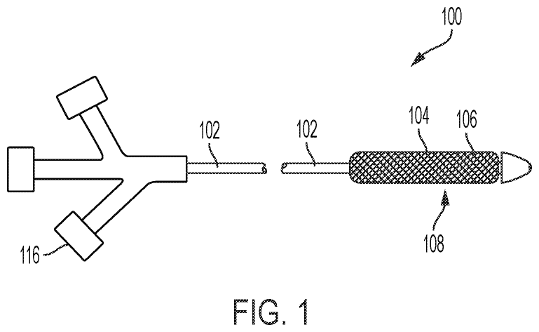

The system shown in FIG. 1 is provided as an example of the various features of the system and, although the combination of those illustrated features is clearly within the scope of invention, that example and its illustration is not meant to suggest the inventive concepts provided herein are limited from fewer features, additional features, or alternative features to one or more of those features shown in the figures. FIG. 1 is a plan view of a delivery system 100 including a catheter 102 with a removable constraint 104, according to some embodiments. As shown in FIG. 1, the removable constraint 104 is configured to constrain an implantable medical device 106 to a delivery configuration. The removable constraint 104 may include one or more fibers or strands 108 arranged about the device 106 to maintain the device 106 and the removable constraint 104 in a constrained or delivery configuration.

The removable constraint 104 is arranged along a length of the device 104. The removable constraint 104 is also circumferentially arranged about the device 106 and may substantially cover the device 106 for delivery. The one or more strands 108 may be arranged within a lumen (not shown) of the catheter 102 and extend toward a proximal end of the catheter 102, which may, in turn, be arranged external to a patient during delivery of the device 106. The one or more strands 108 may include a proximal end 110 that a user may tension in order to release the removable constraint 104 and deploy the device 106.

In certain instances, the one or more strands 108 release such that interlocking portions (e.g., overlapping fibers or knits) sequentially release along the length of the device 106. As is explained in greater detail below, the removable constraint 104 is formed by interlocking together the one or more strands 108 on the device 106. The one or more strands 108 may form release zones 124, 126 including knit rows 130, 132 for releasing the device 106. The device 106 may be a stent, stent-graft, a balloon, prosthetic valve, filter, anastomosis device, occluder or a similar device.

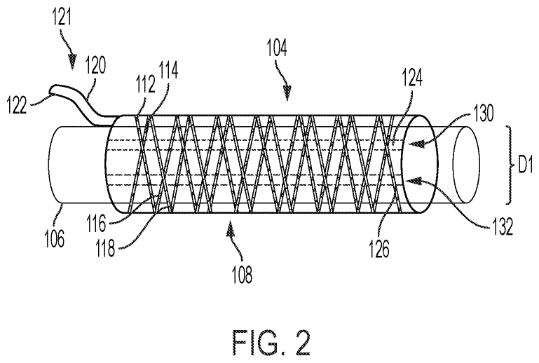

FIG. 2 is a side view of the device 106 including the removable constraint 104, in accordance with an embodiment. As shown, the device 106 includes a delivery diameter D1 and a deployed diameter D2 (not shown) that is larger than the delivery diameter D1. The removable constraint 104 is arranged about the device 106 at the delivery diameter D1. When the removable constraint 104 is removed from the device 106, the device expands to a deployed diameter D2. The deployed diameter D2 is greater than the delivery diameter D1. In some embodiments, the deployed diameter D2 is the diameter of the device 106 when unconstrained. In other embodiments, the deployed diameter D2 is the diameter of the device 106 once the device 106 has been delivered to a target site and has engaged with the lumen wall at the target site.

The device 106 may have a desired deployed diameter D2 from about 5 mm-15 mm, or 6 mm-9 mm, or 6 mm-12 mm, for example, and a delivery diameter D1 that is less than the deployed diameter D2. For example, in some instances, a ratio of the delivery diameter D1 of the device 106 to the deployed diameter D2 (not shown) of the device 106 is less than about 0.3, less than about 0.29, less than about 0.28, less than about 0.27, or less than about 0.26.

As shown in FIGS. 1 and 2, the removable constraint 104 includes at least two interlocking strands 108 in the form of a warp knit. For example, the removable constraint 104 may include a first interlocking strand 112 and a second interlocking strand 114. The first and/or the second interlocking strand(s) 112, 114 may operate, for example, as a first deployment line 120 configured to release the removable constraint 104 and release the device 106 from the delivery diameter D1 to the deployed diameter D2 in response to a deployment force applied to the first deployment line 120. The removable constraint 104 may also include a third interlocking strand 116 and a fourth interlocking strand 118. The third and/or the fourth interlocking strand(s) 116, 118 may operate, for example, as a second deployment line 122 configured to release the removable constraint 104 and release the device 106 from the delivery diameter D1 to the deployed diameter D2 in response to a deployment force applied to the second deployment line 122. It is within the scope of this disclosure to form a removable constraint with two, four, six, eight, or any even number of interlocking strands or any odd number of interlocking strands. In one embodiment, the first and second deployment lines 120, 122 are coupled together to form a unitary deployment segment 121. In some embodiments the first and second deployment lines 120, 122 or the unitary deployment segment 121 includes the proximal end 110 of the one or more strands 108.

The device 104 has a radial force at the delivery diameter D1. The radial force generally refers to the force caused by the device 104 acting on the removable constraint 102 at any point during deployment of the device 104. As discussed above, the interlocking strands 112, 114 are adapted to be removed with a deployment force applied to the deployment line 120. In some instances, the ratio of this radial force of the device 104 to the deployment force applied to the deployment lines 120, 122 is less than about 500:1. In other instances, the ratio of this radial force of the device 104 to the deployment force applied to the deployment lines 120, 122 is less than about 475. In addition, the ratio of this radial force of the device 104 to the deployment force applied to the deployment lines 120, 122 may be less than about 450. In addition, the ratio of this radial force of the device 104 to the deployment force applied to the deployment lines 120, 122 is less than about 425 in other instances. Further, the ratio of the radial force to the deployment force may be between about 10, 20, 30, 40, 50, 100, 200, 300, 400 (or any number in between) and about 500, between about 10, 20, 30, 40, 50, 100, 200, 300, 400 (or any number in between) and about 475, between about 10, 20, 30, 40, 50, 100, 200, 300, 400 (or any number in between) and about 450, or between about 10, 20, 30, 40, 50, 100, 200, 300, 400 (or any number in between) and about 425, for example.

The one or more strands 108, including the interlocking strands 112, 114, 116, 118 in some embodiments, may be formed of various materials, including, for example, polytetrafluoroethylene (PTFE), expanded polytetrafluoroethylene (ePTFE), polyester, polyurethane, fluoropolymers, such as perfluoroelastomers and the like, polytetrafluoroethylene, silicones, urethanes, ultra-high molecular weight polyethylene, aramid fibers, and combinations thereof. Other embodiments for strands 112, 114, 116, 118 can include high strength polymer fibers such as ultra-high molecular weight polyethylene fibers (e.g., Spectra®, Dyneema Purity®, etc.) or aramid fibers (e.g., Technora®, etc.). Generally, any of the foregoing properties may be assessed using ASTM or other recognized measurement techniques and standards, as would be appreciated by a person of ordinary skill in the field.

The various strands 112, 114, 116, 118 may be selected to have specific properties such as strand thickness, strand denier, strand coefficient of friction, strand material, strand treatments, strand coatings, and strand stiffness. Similar to the differing diameter, use of differing strand materials for the strands 112, 114, 116, 118 may increase friction between the first and second interlocking strands 112, 114, 116, 118 to help maintain the device 104 in the delivery configuration. Each of the various strands may be selected to include the same strand properties or different strand properties based on the application in which the removable constraint will be used. It is recognized that the properties of the strands 112, 114, 116, 118 may also be altered by treatments, configurations, and alterations, in addition to material selection. For example, the strands may include fillers or core materials, may be surface treated by etching, vapor deposition, or coronal or other plasma treatment, among other treatment types, including being coated with suitable coating materials.

With further reference to FIG. 2, the removable constraint 104 may include a first release zone 124 and a second release zone 126. The first release zone 124 may be defined along at least a portion of a longitudinal length of the removable constraint 104. The second release zone 126 may be defined along at least a portion of the longitudinal length of the removable constraint 104. In some embodiments the first and second release zones 124, 126 are coextensive along the longitudinal length of the removable constraint. The first and second release zones 124, 126 may be spaced relative to one another around the circumference or outer dimension of the removable constraint 104. In some embodiments the first and second release zones 124, 126 may be spaced across from each other relative to the circumference or outer dimension of the removable constraint 104. In other embodiments, the first and second release zones 124, 126 are disposed on a first face of the removable constraint 104. In one embodiment, the first release zone 124 is spaced from and proximate to the second release zone 126 about the outer dimension or circumference of the removable constraint 104 and is coextensive with the second release zone 126 along the longitudinal length of the removable constraint. In accordance with an embodiment, the first and second release zones 124, 126 may be positioned at different locations about the circumference of the removable constraint 104. The release zones may be spaced about the circumference from about 10 degrees to about 180 degrees, from about 20 degrees to about 30 degrees, from about 30 degrees to about 45 degrees, from about 45 degrees to about 60 degrees, from about 60 degrees to about 75 degrees, from about 75 degrees to about 90 degrees, from about 90 degrees to about 105 degrees, from about 105 degrees to about 120 degrees, from about 120 degrees to about 135 degrees, from about 135 degrees to about 145 degrees, from about 145 degrees to about 160 degrees, from about 160 degrees to about 180 degrees. For example, the release zones can be spaced approximately 180 degrees apart from one another, approximately 90 degrees apart from one another, approximately 60 degrees apart from one another, or any other distance as desired. The release zones may be spaced approximately equidistant about the circumference of the removable constraint 104, or they may be offset as desired.

As can be seen in FIG. 2, the removable constraint 104 may include two release zones 124, 126. The first release zone 124 may comprise a first knit row 130. The second release zone 126 may comprise a second knit row 132. The knit rows 130, 132 may be formed by the interlocking of the various strands 112, 114, 116, 118 previously discussed. For example, the first knit row 130 may comprise portions of the removable constraint 104 at which the first and second strands 112, 114 are interwoven. The second knit row 132 may comprise portions of the removable constraint 104 at which the third and fourth strands 116, 118 are interwoven. In some embodiments the first, second, third, and fourth strands 112, 114, 116, 118 are all interwoven to form the removable constraint 104, wherein the first release zone 124 includes a first knit row 130 formed at portions of the first and second strands 112, 114 where the first and second strands 112, 114 are interwoven and the second release zone 126 includes a second knit row 132 formed at portions of the second and third strands 116, 118 where the third and fourth strands 116, 118 are interwoven. It is understood that the removable constraint is not limited to only two release zones, but any number of release zones may be implemented, including a third release zone formed from a third set of strands, a fourth release zone formed from a fourth set of stands, or any number of release zones formed from corresponding sets of strands.

The removable constraint 104 may include a first deployment line 120 and a second deployment line 122 configured to deploy the device 106 by disengaging the removable constraint 104 from the device 106. This may occur via an unravelling of the knit rows 130, 132 of the first and second release zones 124, 126, and consequently portions of the body of the removable constraint 104. In one embodiment, the first deployment line 120 extends from the first knit row 130 and is engaged with the first knit row 130 such that the first deployment line 120 is operable to disengage or unravel at least a first portion of the first knit row 130. The first deployment line 120 may include portions of each of the strands comprising the first knit row 130, for example, the first deployment line 120 may comprise the first and second strands 112, 114. Similarly, the second deployment line 122 extends from the second knit row 132 and is engaged with the second knit row 132 such that the second deployment line 122 is operable to disengage or unravel at least a first portion of the second knit row 132. The second deployment line 122 may include portions of each of the strands comprising the second knit row 132, for example, the second deployment line 122 may comprise the third and fourth strands 116, 118. Each of the knit rows 130, 132 is operably to sequentially unravel as the respective deployment lines 120, 122 are engaged.

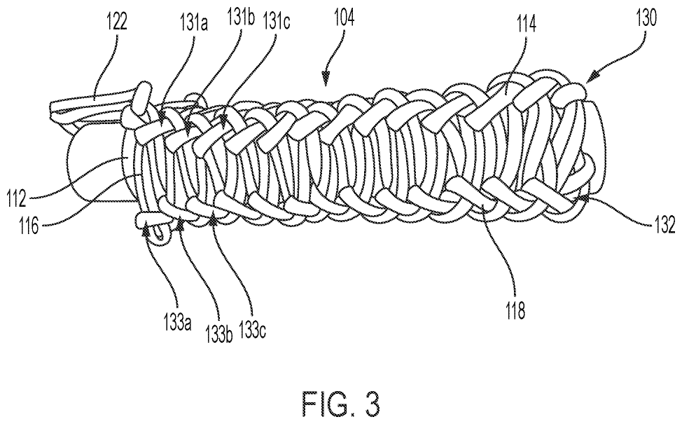

An example is shown in FIG. 3 in which the first knit row 130 may include a first knit 131a, a second knit 131b, a third knit 131c, and so forth. The first deployment line 122 may be an extension of or the free end of the first interwoven strand 112 that forms the first knit row 130 with portions of the second interwoven strand 114. In some embodiments, the first deployment line is integrally formed with either the first or second strand 112, 114. When the first deployment line 122 is engaged or tensioned such that the first deployment line 122 advances away from the removable constraint 104, the first knit 131a unravels. As the first deployment line 122 continues to be tensioned and advanced away from the removable constraint 104, the second knit 131b unravels. This occurs until the first knit row 130 has partially or fully unraveled. A similar pattern occurs and features may be included with regard to the second knit row 132 and its respective knits 133a, 133b, 133c. In some embodiments, the first deployment line 122 may include knit portions of the first and second strands 112, 114.

In some embodiments, the interwoven strands 112, 114, 116, 118 are knit such that the first and second knit rows 130, 132 are deployed substantially simultaneously in order to facilitate the unraveling of the knit rows 130, 132 and more specifically the strands at the knit rows 130, 132 of the removable constraint 104. Because the strands 112, 114, 116, 118 are all interwoven, when the first or second knit row 130, 132 is advanced or unraveled at a different rate than the other knit row, the strands forming the other knit row interfere with the proper unraveling of the former knit row. This occurs by the binding or restriction of the deployment line at one knit row until the other knit row has been sufficiently advanced to release the deployment line from the other knit row. Because all of the strands 112, 114, 116, 118 may be interwoven, this restriction of the deployment lines 120, 122 may occur when either of the knit rows 130, 132 are unraveled disproportionately relative to the other.

For example, if the first deployment line 122 is tensioned such that the first knit 131a is unraveled and the first deployment line 122 continues to be tensioned and the corresponding first knit 133a of the second knit row 132 has not been unraveled, the strands 116, 118 of the second knit row 132 may interfere with the unraveling of the second knit 131b. In this example, the first strand 112 of the first knit row 130 may be interwoven with the strands 116, 118 of the second knit row 132 such that the first knit row 130 is unable to advance until the first strand 112 and/or second strand 114 is/are released from the strands 116, 118 of the second knit row 132 via the release or unraveling of the corresponding first knit 133a of the second knit row 132. However, if the corresponding first knit 133a of the second knit row 132 is unraveled, the first strand 112 and/or second strand 114 may be freed from the corresponding first knit 133a such that tension across the first deployment line 122 may initiate deployment of second knit 131b, which is then unraveled. It is noted that the converse may also be true, such that the second deployment line 124 may be restricted from unraveling the second knit row 132 if corresponding knits of the first knit row 130 are undeployed.

In some embodiments, the corresponding knits of the first and second knit rows 130, 132 must be deployed before the subsequent knit in the knit row can be sequentially deployed. In other embodiments, the strands 112, 114, 116, 118 are interwoven such that the subsequent knits may be deployed when the corresponding knits of the other knit row are not deployed. In yet another embodiment, the strands 112, 114, 116, 118 are interwoven such that a subsequent knit (e.g., second knit 131b) in a knit row may deploy when a corresponding knit (e.g., corresponding first knit 133a) is undeployed; however, a knit subsequent to the subsequent knit (e.g., third knit 131c) may be restricted when the corresponding knit is undeployed. The pattern for interweaving the various strands may be altered to provide various interactions between the knit rows for restricting unraveling. For instance, a knit row may advance or be unraveled at two knits, three knits, four knits, or five knits beyond the unravelling of the corresponding intact knits of the other knit row based on the weave or knit pattern. By varying how far a knit row may advance past the other knit row in unraveling, the constraining force and/or the precision of deployment may be varied when delivering and deploying a device 106.

In some embodiments, it may be understood that the removable constraint 104 may be formed as two sleeves. The first sleeve may be formed of the first and second strands 112, 114 and the second sleeve may be formed of the third and fourth strands 116, 118. The two sleeves and their respective strands may be understood to be overlaid and intertwined or interwoven such they are coaxial and may resist deployment when one sleeve is deployed further than the other; however, each of the sleeves forms knit rows that are independent from the other knit row of the corresponding sleeve. This means that the knit row is formed of the strands of the sleeve and not the strands of the other sleeve. But, as previously described corresponding knit rows may interfere or restrict deployment of the other knit row when they are not deployed along a substantially similar length of the removable constraint by binding or restricting fibers of the other sleeve.

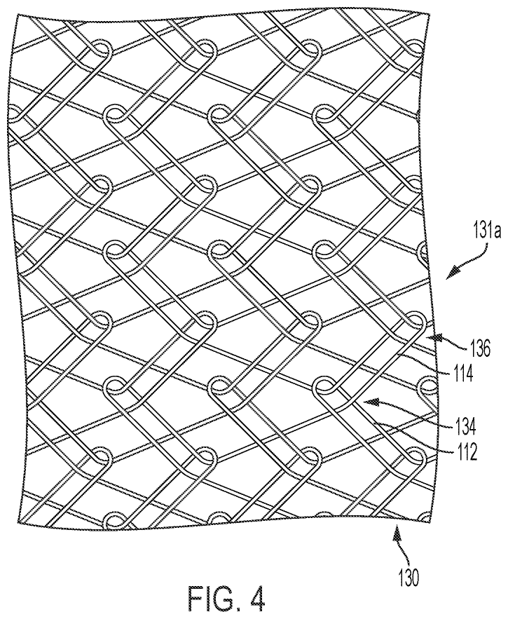

Referring now to FIG. 4, a schematic view of interlocking strands of the removable constraint 104 (FIGS. 1-3) is provided in accordance with an embodiment. The interlocking strands (e.g., the first and second interlocking strands 112, 114, as shown) are generally interwoven with one another to form at least one knit row 130. As shown, the knit row 130 is formed of interlocking loops formed from the first and second interlocking strands 112, 114. For example, a first knit 131a is formed of interlocking loops 134 of the first interlocking strand 112 interwoven with second interlocking loops 136 formed by the second interlocking strand 114. This interlocking, looped configuration allows for release of the removable constraint 104 by applying the deployment force (releases knit force) to the deployment line.

Turning now to a discussion of the methods for making and using a removable constraint, the method of deploying a device with the disclosed removable constraint is provided. As previously discussed, a medical device may include an expandable device capable of expanding and contracting to various diameters, including a first constrained diameter D1 and a second expanded diameter D2. The expandable device may be maintained in a constrained configuration by a removable constraint, the removable constraint comprising a plurality of strands interwoven to form a first release zone and a second release zone, each comprising knit rows with a plurality of knits. In some embodiments, at least one deployment line extends from each of the release zones.

The method of deploying a medical device may include delivering the device to the treatment site intravenously. The expandable medical device is positioned in a patient, wherein the expandable medical device is constrained by a removable constraint in a compressed configuration. The first release zone is configured to disengage the removable constraint from the expandable medical device via the first deployment line operable to activate the first release zone. The second release zone is configured to disengage the removable constraint from the expandable medical device via the second deployment line operable to activate the second release zone. A user may retain portions of the first deployment line and the second deployment line remote from the expandable medical device, i.e., outside of the intravenous access site. The user may then apply sufficient force to the first deployment line and the second deployment lines to activate the first release zone and the second release zone. As the first and second release zones are activated, the medical device may be released from the removable constraint and deploy within the anatomy of the patient. Thus, as the release zones are activated, the removable constraint is at least partially deconstructed and the expandable device is able to expand from the constrained diameter to a deployed diameter.

In some embodiments, the method includes simultaneously applying sufficient force to the free ends of the first deployment line and the second deployment line, or applying such force in relatively close temporal sequence. As discussed above, this step may be important when the plurality of strands are interwoven, such that the knits of the deployment zones interfere with the release or deployment of the corresponding knits. As the deployment lines are activated, they may be translated away from the delivery site. The plurality of lines may be removed via the catheter.

The disclosure also relates to a method of manufacturing an expandable medical device. The method may include compressing an expandable member radially inward to a first compressed diameter. A plurality of strands including a first deployment line and a second deployment line may be interwoven to form a removable constraint. The removable constraint may be interwoven such that a first deployment zone and a second deployment zone are formed of two knit rows as discussed previously. The method may include providing a first free end of the first deployment line such that at least a portion of the first deployment line extends away from the removable constraint and providing a second free end of the second deployment line such that at least a portion of the second deployment line extends away from the removable constraint. The first free end and the second free end may be operable to deconstruct the removable constraint when deployed substantially simultaneously. The method may also include coupling the first free end and the second free end such that they form a unitary deployment segment.

The method of manufacturing may also include weakening at least one of the strands comprising the covering member, such that the strand is operable to break. When at least one of the strands breaks, the deployment of the corresponding knit row may begin deployment. Thus, in some embodiments, one strand from each of the knit rows may be weakened in order to facilitate the breaking of a strand from each of the knit rows. In some embodiments, the weakened strands comprise the deployment lines of the knit rows. In other embodiments, the unweakened strands comprise the deployment lines of the knit rows, such that when the unweakened strands are tensioned, the unweakened strands stay intact and the weakened strands break, which initiates deployment of the knit rows. The knit rows may initially be tensioned in order to provide the first break. Tension may then be applied to the unbroken, unweakened strands in order to continue deployment of the knit rows as the knit rows unravel under tension due to the unraveling of the knit row. The knit row continues to unravel due to the break in the strand which allows for the knit row to unravel or separate. The weakening of the strands may be accomplished by scoring, cutting, treating, or otherwise compromising the strand such that the strands may break under predetermined circumstances such as tensioning.

As part of the manufacturing method, deployment of the covering member may be initiated by breaking at least one strand from each of the knit rows. The covering member may be deployed to a desired length relative to the expandable member. For example, the covering member may be knit to a length longer than the expandable member. The covering member may be activated such that deployment is initiated. The covering member may be partially deployed until a desired length of the covering member is achieved, such as a length where the covering member is surrounding the expandable member and does not extend beyond the longitudinal ends of the expandable member. At this point, the deployment may be discontinued until the medical device is prepared for deployment at the target site. It is understood that the covering member may be partially deployed to any desirable length. The partially deployed covering member that is constraining the expandable member may then be prepared for packaging, use, installation on a catheter, or any other desired action.

In some embodiments, the method of includes performing the step of compressing the expandable member simultaneously with the step of compressing the expandable member such that the plurality of strands provide a compressive force to the expandable member as the plurality of strands are interwoven about the expandable member.

In some embodiments, the covering member may be woven on a mandrel. Once the covering member is woven, and in some embodiments partially deployed, the covering member may be removed from the mandrel and applied over a radially compressed implantable medical device.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A medical device comprising:
an expandable member configured to radially expand from a first diameter toward a second diameter; and
a knit constraining member positioned around the expandable member to constrain the expandable member at the first diameter, the knit constraining member having a first release zone configured to disengage the knit constraining member from the deployable member, a first deployment line operable to activate the first release zone, a second release zone configured to disengage the knit constraining member from the deployable member, and a second deployment line operable to activate the second release zone,
wherein the constraining member is configured to disengage from the deployable member by substantially simultaneously tensioning the first deployment line and the second deployment line.

2. The medical device of claim 1, wherein the constraining member comprises a first body strand and a second body strand interwoven to form the first deployment line and a third body strand and a fourth body strand interwoven to form the second deployment line.

3. The medical device of claim 2, wherein the first body strand, the first deployment line, the second body strand, and the second deployment line are all interwoven.

4. The medical device of claim 3, wherein the first body strand, the first deployment line, the second body strand, and the second deployment line are warp knit.

5. The medical device of claim 2, wherein the first body strand and the first deployment line form a first plurality of knits along at least a first portion of a longitudinal length of the constraining member, and wherein the second body strand and the second deployment line form a second plurality of knits along at least a second portion of the longitudinal length of the constraining member.

6. The medical device of claim 5, wherein the first release zone comprises the first plurality of knits and the second release zone comprises the second plurality of knits.

7. The medical device of claim 6, wherein the first plurality of knits are sequentially unraveled when a first threshold tension is applied across the first deployment line and wherein the second plurality of knits are sequentially unraveled when a second threshold tension is applied across the second deployment line.

8. The medical device of claim 7, wherein the first deployment line is interwoven with the second deployment line and the second body strand such that the second plurality of knits are operable to unravel when corresponding knits of the first plurality of knits are unraveled by advancing the first deployment line away from the first release zone.

9. The medical device of claim 7, wherein the second deployment line is interwoven with the first deployment line and the first body strand such that the first plurality of knits are operable to unravel when corresponding knits of the second plurality of knits are unraveled by advancing the second deployment line away from the second release zone.

10. The medical device of claim 1, wherein the first deployment line and the second deployment line each include free ends, wherein the free ends of the first deployment line and the second deployment line are coupled to form a unitary deployment segment.

11. The medical device of claim 1, wherein the first release zone and the second release zone are configured to deploy substantially simultaneously.

12. The medical device of claim 1, wherein the first deployment line, the first body strand, the second deployment line, and the second body strand comprise expanded polytetrafluorethylene.

13. The medical device of claim 1, where the first release zone and the second release zone are configured to provide resistance to an outward expansion of the deployable member when undeployed.

14. The medical device of claim 1, wherein the implantable medical device has a radial force at a delivery diameter of the deployable member, and wherein the first deployment line and the second deployment line are configured to be removed with a deployment force applied to the first deployment line and the second deployment line; and wherein a ratio of the radial force to the deployment force is between about 100 and about 500.

15. The medical device of claim 1, wherein a ratio of deployable member delivery diameter to deployment diameter is less than 0.3.

16. An expandable medical device comprising:
a deployable member; and
a removable constraint including a plurality of interlocking strands in the form of a warp knit,
wherein the removable constraint is positioned axially exterior to the deployable member and restrains the deployable member in a radially compressed orientation,
wherein the plurality of interlocking strands includes a first deployment line and a second deployment line, and
wherein the removable constraint is configured to be remotely removed when force is applied to the first deployment line and the second deployment line.

17. The expandable medical device of claim 16, wherein the removable constraint includes a first release zone and a second release zone.

18. The expandable medical device of claim 17, wherein the first release zone is formed at least partially of the first deployment line, and wherein the second release zone is formed at least partially of the second deployment line.

19. The expandable medical device of claim 18, wherein the first release zone is configured to sequentially unravel when tension is applied to the first deployment line, and wherein the second release zone is configured to sequentially unravel when tension is applied to the second deployment line.

20. The expandable medical device claim 16, wherein the removable constraint is configured to be removed from the deployable member by substantially simultaneously applying force to the first deployment line and the second deployment line.

21. A method of deploying a medical device, comprising:
positioning an expandable medical device in a patient, wherein the expandable medical device is constrained by a constraining member in a compressed configuration, wherein the constraining member includes a first release zone configured to disengage the constraining member from the expandable medical device, a first deployment line operable to activate the first release zone, a second release zone configured to disengage the constraining member from the expandable medical device, and a second deployment line operable to activate the second release zone;
retaining proximal portions of the first and second deployment lines remote from the expandable medical device; and
applying sufficient force to the proximal portions of the first and second deployment lines to activate the first release zone and the second release zone.

22. The method of claim 21, wherein the step of applying sufficient force to the proximal portions of the first and second deployment lines includes simultaneously applying sufficient force to the proximal portions of the first and second deployment lines.
